(19) United States
(12) Patent Application Publication
(10) Pub. No.: US 2023/0392107 A1
(43) Pub. Date: Dec. 7, 2023

(54) PRODUCT RECOVERY FROM FERMENTATION

(71) Applicant: LanzaTech, Inc., Skokie, IL (US)

(72) Inventors: Paul Alvin Sechrist, South Barrington, IL (US); Robert John Conrado, Washington, DC (US); Elham Ebrahimiaqda, Minneapolis, MN (US)

(21) Appl. No.: 17/804,948

(22) Filed: Jun. 1, 2022

(51) Int. Cl. C12M 1/00; C07C 29/86; C07C 29/80

(52) U.S. Cl. CPC C12M 47/10; C07C 29/86; C07C 29/80

(57) ABSTRACT

The disclosure is directed to a method and a system for recovering at least one organic product from fermentation broth. The disclosure relates to the use of liquid-liquid extraction and/or distillation to recover at least one organic product from a fermentation broth. In an embodiment, the recovery of the product is completed in a manner that minimizes stress on the microbial biomass, such that it remains viable, at least in part, and may be recycled and reused in the fermentation process, with minimal water extraction which results in increased efficiency in the fermentation process.

### FIELD

The disclosure relates to methods and systems for recovering one or more organic products from microbial fermentation processes. In particular, the disclosure relates to recovering organic products, such as ethanol, from a fermentation broth.

### BACKGROUND

Carbon dioxide (CO₂) accounts for about 76% of global greenhouse gas emissions from human activities, with methane (16%), nitrous oxide (6%), and fluorinated gases (2%) accounting for the balance (the United States Environmental Protection Agency). Reduction of greenhouse gas emissions, particularly CO₂, is critical to halting the progression of global warming and the accompanying shifts in climate and weather. Recently, gas fermentation has emerged as a platform for the biological fixation of C1-carbon sources. The C1-carbon source refers to a one-carbon molecule that serves as a partial or sole carbon source for a microorganism in gas fermentation. For example, a C1-carbon source may comprise at least one of CO, CO₂, CH₄, CH₃OH, or any combinations thereof. Typically, a substrate is a gaseous carbon and/or energy source comprising at least the C1-carbon source. The substrate may further comprise other non-carbon components, such as hydrogen (H₂) or nitrogen (N₂). The C1-carbon source and/or substrate may be a waste gas obtained as a by-product of an industrial process or from another source, such as combustion engine exhaust fumes, biogas, landfill gas, direct air capture, or electrolysis. The C1-carbon source and/or substrate may be syngas generated by pyrolysis, torrefaction, or gasification.

In particular, C1-fixing microorganisms have been demonstrated as biocatalysts to convert C1-carbon source and/or substrate into valuable products such as alcohols. The gas fermentation process utilizes a culture of one or more C1-fixing microorganisms to produce fermentation broth comprising one or more products from a C1-carbon source and/or substrate. The production of such products may be limited, for example, by slow microbial growth, limited gas consumption, sensitivity to toxins, or diversion of carbon substrates into undesired by-products. The accumulation of products and by-products can reduce the production efficiency of the gas fermentation process. These products and by-products should be removed at an effective rate to prevent accumulation. If not removed at an effective rate, these products may have inhibitory and/or toxic effects on the C1-fixing microorganisms. If the products accumulate to the point that the C1-fixing microorganisms cannot survive, then the fermentation process may have to be stopped and restarted. Prior to being restarted, the fermenters often require cleaning. This can be a time-consuming process.

For continuous gas fermentation processes, the product and by-products should be continuously removed from the fermentation broth while the remaining fermentation broth components should be recycled to the bioreactor. The separation of desired products from the fermentation broth involves multiple steps and processing high volumes of water present in the fermentation broth through these multiple steps is challenging. Benefits may be realized through selecting techniques and organizing the multiple steps so that the processing and removal of water from the broth or other streams during product recovery could be minimized. However, C1-fixing microorganisms present in fermentation broth are unlikely to survive the high temperatures required for conventional distillation, other separation techniques such as vacuum distillation or filtration methods have been employed to recycle viable microorganisms to the bioreactor. However, in traditional filtration methods, particulate matter can build up in or on the filter over time, leading to a reduction in the filtrate flux and cleaning and/or replacing the filter. Further, when the C1-fixing microorganisms are no longer viable and removed from the bioreactor, conventional separation techniques require significant amounts of water and added nutrient media also be removed along with the non-viable microorganisms. The overall system becomes depleted of water and nutrients which then should be replenished at added expense. More advantageous techniques are needed.

Further, concentrations of organic products, such as C1-C18 alcohols, in a sugar-based fermentation broth is about 8-14 wt. %. However, in a gas fermentation process, the organic products such as C1-C18 alcohols in the fermentation broth are normally present at much lower concentrations, such as up to about 3 wt. %. Separation and recovery of the organic products from aqueous gas fermentation effluent after removal and recycling of viable microorganisms requires higher energy input through conventional distillation as compared to the recovery of organic products of sugar-based fermentation which are present in higher concentrations. Moreover, recovery of one part of organic products is typically followed by treating and recycling up to three parts of wastewater, resulting in higher costs to maintain proper pH levels of 4 to 7 in the wastewater treatment unit and higher initial CAPEX for a larger wastewater treatment unit. Reduced volumes of wastewater, and thus reduced need for wastewater treatment, has the added benefit of potentially limiting the opportunity for introducing foreign bacteria into the system. Such foreign bacteria may produce undesired products or prove harmful to the intended C1-fixing microorganism of the process.

Accordingly, there remains a need for an effective method for recovering at least one organic product from gas fermentation broth with minimal water removal from the broth, while simultaneously assisting the viability of the C1-fixing microorganisms in the fermentation broth.

### SUMMARY

This disclosure provides a process and a system for recovering at least one organic product from fermentation broth. A fermentation broth comprising water, media, biocatalyst, and the at least one organic product is generated in a bioreactor. The fermentation broth is separated by liquid-liquid extraction using a solvent to generate a raffinate stream enriched in water and media, and an extract stream enriched in at least one organic product and solvent. The extract stream is reduced in water and media. The extract stream is passed to a distillation unit where it is separated into an overhead stream and a bottoms stream. The overhead stream is enriched in at least one organic product and reduced in water and media and the bottoms stream is enriched in the solvent, water, and media. The raffinate stream and at least a first portion of the bottoms stream may be recycled to the bioreactor. At least a second portion of the bottoms stream is passed to the liquid-liquid extraction process. The solvent for the liquid-liquid extraction process is methyl isobutyl ketone, ethyl isobutyl ketone, or any combination thereof and at least one organic product is selected from C1 to C18 alcohols and carboxylic acids.

In one embodiment, the overhead stream may be passed to a purification process, such as distillation, pressure swing adsorption (PSA), or membrane filtration process, for separation into a product stream comprising at least one organic product and an effluent stream enriched in water and media. At least a portion of the effluent stream may be recycled to the bioreactor, to a wastewater treatment unit, or to both. A treated water stream may be generated in the wastewater treatment unit and the treated water stream may be recycled to the bioreactor as a make-up water supply. The fermentation broth may comprise an undesired component, an amount of the undesired component may be removed from the first portion of the bottoms stream before recycling it to the bioreactor. The fermentation broth and the extract stream and the overhead stream may further comprise fusel oils. A fusel oil stream may be separated from the overhead stream in the purification unit.

At least a portion of the biocatalyst from the fermentation broth may be separated to form a biocatalyst enriched broth before separating the fermentation broth by liquid-liquid extraction. At least a portion of the biocatalyst enriched fermentation broth may be recycled to the bioreactor. At least a portion of the biocatalyst may be dried and recovered from the biocatalyst enriched fermentation broth.

The disclosure further provides an apparatus for recovering at least one organic product from fermentation broth. The apparatus comprises a bioreactor comprising at least a fermentation broth conduit; a liquid-liquid extraction unit in fluid communication with the fermentation broth conduit, the liquid-liquid extraction unit comprising a raffinate conduit and an extract conduit; a distillation unit in fluid communication with the extract conduit, the distillation unit comprising an overhead conduit and a bottoms conduit in fluid communication with the liquid-extraction unit.

In an embodiment, the system may further comprise a purification unit in fluid communication with the overhead conduit, the purification unit comprising a product conduit, an effluent conduit, and a fusel oil conduit. A separator may also be in fluid communication with the fermentation broth conduit, and the liquid-liquid extraction unit. A wastewater treatment unit may be in fluid communication with the effluent conduit. The raffinate conduit may be in fluid communication with the bioreactor.

The system may further comprise a removal unit in fluid communication with the wastewater treatment unit, the effluent conduit, and the bottoms conduit. The removal unit is further in fluid communication with the bioreactor. The system may further comprise a drier in fluid communication with the bioreactor. The bioreactor may further comprise at least one substrate conduit, at least one media conduit, and an off-gas conduit.

### BRIEF DESCRIPTION OF THE DRAWINGS

The Figure(s) has been simplified by the deletion of a large number of apparatuses customarily employed in a process of this nature, such as vessel internals, temperature and pressure controls systems, flow control valves, recycle pumps, and the like. which are not specifically required to illustrate the performance of the invention. Furthermore, the illustration of the process of this invention in the embodiment of a specific drawing is not intended to limit the disclosure to specific embodiments. Some embodiments may be described by reference to the process configuration shown in the figure, which relate to both apparatus and processes to carry out the disclosure. Any reference to a process step includes reference to an apparatus unit or equipment that is suitable to carry out the step, and vice-versa.

FIG. 1 shows a flow scheme of a process and apparatus for recovering at least one organic product from fermentation broth.

FIGS. 2a-2c show graphs showing the selectivity and distribution coefficient (K-EtOH) of ethanol versus the composition by weight of the methyl isobutyl ketone (MIBK) solvent at 10° C., 25° C., and 50° C. respectively.

### DETAILED DESCRIPTION

There is an overall need to reduce the amount of water that is removed from broth during continuous gas fermentation and at the same time maintain low utility and capital investment requirements for product separation and wastewater treatment. C1-C3 alcohols may be separated by simple distillation when their respective vapor pressures are higher than the vapor pressure of water in the fermentation broth, while C4 and higher alcohols have a lower vapor pressure (higher boiling point) than the vapor pressure of water in the fermentation broth. In order to separate C4 and higher alcohols from the fermentation broth, water is needed to boil off, and that amount of energy would be prohibitive, so liquid-liquid extraction becomes a leading alternative. Incorporating liquid-liquid extraction into the overall gas fermentation process flow scheme allowed for low-cost separation of the desired product as well as reduction of costs involved in wastewater treatment. Furthermore, opportunities for the microbial upset to the bioreactors are minimized. The disclosure is explained in terms of a continuous gas fermentation process but may be applied to batch gas fermentation processes or other fermentation processes where the substrate is not a gas stream such as sugar-based fermentation processes.

The gas fermentation bioreactor includes a culture of one or more C1-fixing microorganisms that have the ability to produce one or more products from a C1-carbon source. “C1” refers to a one-carbon molecule, for example, CO, CO₂, CH₄, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. In an embodiment, the C1-carbon source comprises one or both of CO and CO₂. “Substrate” refers to a carbon and/or energy source for the microorganism of the disclosure. Typically, the substrate is gaseous and comprises a C1-carbon source, for example, CO, CO₂, and/or CH₄. The substrate may further comprise other non-carbon components, such as H₂, N₂, or electrons. Although the substrate is typically gaseous, the substrate may also be provided in alternative forms. For example, the substrate may be dissolved in a liquid saturated with a C1-carbon source gas using a microbubble dispersion generator. By way of further example, the substrate may be adsorbed onto a solid support.

The substrate and/or C1-carbon source may be a waste gas obtained as a by-product of an industrial process or from another source, such as combustion engine exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane, and in certain embodiments, the substrate and/or C1-carbon source may be a non-waste gas. Although the substrate is typically gaseous, the substrate may also be provided in alternative forms.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas such as when biogas is added to enhance gasification of another material. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture waste and forest waste.

The substrate and/or C1-carbon source may be a gas stream comprising methane. Such a methane containing gas may be obtained from fossil methane emissions such as during fracking, wastewater treatment, livestock, agriculture, and municipal solid waste landfills. It is also envisioned that the methane may be burned to produce electricity or heat and the C1 by-products may be used as the substrate or carbon source.

In certain embodiments, the industrial process is selected from ferrous metal products manufacturing, such as steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as naturel gas, oil, or coal fired boilers or heaters, and gas turbine exhaust. In these embodiments, the substrate and/or C1-carbon source may be captured from the industrial process before it is emitted into the atmosphere, using any known method.

The gas fermentation process is a platform for the biological fixation of carbon in the gas streams comprising carbon dioxide (CO₂), carbon monoxide (CO), hydrogen (H₂), or methane (CH₄) using C1-fixing microorganisms as biocatalysts to convert the substrate and/or C1-carbon source into valuable products such as ethanol or other alcohols. A C1-fixing microorganism is a microorganism that has the ability to produce one or more products from a C1-carbon source. Typically, the microorganism of the disclosure is a C1-fixing bacterium. A “microorganism” or “biocatalyst” is a microscopic organism, especially a bacterium, archea, virus, or fungus. The microorganism of the disclosure is typically a bacterium. As used herein, recitation of “microorganism” should be taken to encompass “bacterium”. “Viable microorganisms” or “viability of the microbial biomass” and the like refers to the ratio of microorganisms that are alive, capable of living, developing, or reproducing to those that are not. The disclosure may be designed so that the viability of the microbial biomass is maintained at a minimum viability.

The microorganisms in the bioreactor may be modified from a naturally occurring microorganism. A “parental microorganism” is a microorganism used to generate a microorganism of the disclosure. The parental microorganism may be a naturally occurring microorganism, known as a wild-type microorganism or a microorganism that has been previously modified, known as a mutant or recombinant microorganism. The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In an embodiment, the parental microorganism is Clostridium autoethanogenum LZ1561, which was deposited on Jun. 7, 2010, with Deutsche Sammlung von Mikroorganismen und Zellkulturen GmbH (DSMZ) located at Inhoffenstraße 7B, D-38124 Braunschweig, Germany on Jun. 7, 2010, under the terms of the Budapest Treaty and accorded accession number DSM23693. This strain is described in International Patent Publication No. WO 2012/015317.

The microorganism of the disclosure may be cultured with the substrate and/or C1-carbon source in gas fermentation to produce one or more products. For instance, the microorganism of the disclosure may produce or may be engineered to produce ethanol (WO 2007/117157), acetate (WO 2007/117157), butanol (WO 2008/115080 and WO 2012/053905), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2014/0369152), chorismate-derived products (WO 2016/191625), 3-hydroxybutyrate (WO 2017/066498), 1,3-butanediol (WO 2017/066498) 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to 2-phenylethanol (WO 2021/188190).

The microorganism of the disclosure may be engineered to produce products at a certain selectivity or at a minimum selectivity. In one embodiment, a target product accounts for at least about 5%, 10%, 15%, 20%, 30%, 50%, 75%, or 95% of all fermentation products produced by the microorganism of the disclosure. In one embodiment, the target product accounts for at least 10% of all fermentation products produced by the microorganism of the disclosure, such that the microorganism of the disclosure has a selectivity for the target product of at least 10%. In another embodiment, the target product accounts for at least 30% of all fermentation products produced by the microorganism of the disclosure, such that the microorganism of the disclosure has a selectivity for the target product of at least 30%.

Fermentation, including “fermenting”, “fermentation process” or “fermentation reaction” and the like, encompasses both the growth phase and product biosynthesis phase of the microorganisms. Continuous fermentation allows for extended operations since product and/or metabolite is extracted during fermentation. One example of a technique to remove product and/or metabolite is vacuum distillation. The disclosure is most advantageous in a continuous fermentation process but is envisioned as applicable to batch fermentation as well. During batch fermentation, the bioreactor is filled with raw material, i.e., the carbon source, along with microorganism biocatalyst and the products and/or metabolites remain in the bioreactor until fermentation is completed. After batch fermentation is completed, the products are extracted, and the bioreactor is cleaned before the next batch is started.

The fermentation culture is generally maintained in an aqueous culture medium that contains nutrients, vitamins, and/or minerals sufficient to permit growth of the microorganism. In an embodiment, the aqueous culture medium is an anaerobic microbial growth medium, such as a minimal anaerobic microbial growth medium. Suitable media are well known in the art. The term “fermentation broth” or “broth” is intended to encompass the mixture of components including the nutrient media, the culture of one or more microorganisms, water, and one or more products. The terms microorganism, bacteria, and biocatalyst are used interchangeably throughout the disclosure.

The fermentation process should be carried out under appropriate conditions for the production of the target product. Typically, the fermentation is performed under anaerobic conditions. Reaction conditions to consider include pressure or partial pressure, temperature, gas flow rate, liquid flow rate, media pH, media redox potential, agitation rate if using a continuous stirred tank reactor, inoculum level, maximum gas substrate concentrations to ensure that gas in the liquid phase does not become limiting, and maximum product concentrations to avoid product inhibition. In particular, the rate of introduction of the substrate or components within the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be conducted at pressures higher than atmospheric pressure. Also, since a given gas conversion rate is, in part, a function of the substrate retention time and retention time dictates the required volume of a bioreactor, the use of pressurized systems can reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used.

The fermentation broth is generated in a bioreactor which includes a fermentation device consisting of one or more vessels and/or towers or piping arrangements. Example of bioreactors include continuous stirred tank reactor (CSTR), immobilized cell recycles (ICR), trickle bed reactor (TBR), bubble column, gas lift fermenter, static mixer, a circulated loop reactor, a membrane reactor, such as a hollow fibre membrane bioreactor (HFM BR) or other unit or other device suitable for gas-liquid contact. The reactor may be adapted to receive a gaseous substrate comprising CO and/or CO₂, or H₂ or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

The present disclosure provides a method and a system that helps in recovering at least one organic product from fermentation broth with minimal water removal from the bioreactor while assisting the viability of the C1-fixing microorganisms in the fermentation broth.

The fermentation broth generated in the bioreactor may be passed to an optional separator. At least a portion of a biocatalyst is separated from the fermentation broth to generate a biocatalyst enriched fermentation broth and a biocatalyst depleted fermentation broth. The separation is carried out so as to ensure the viability of the microbial biomass so that the biocatalyst enriched fermentation broth may be recycled to the bioreactor and again be utilized for gas fermentation. If desired, the viability of the microbial biomass may be measured using any suitable means. Known separation techniques may be employed in the separator. For example, the separator may employ a centrifugation technique or the separator may employ a filtration technique such as membrane filtration. In membrane filtration, the membrane retains biocatalyst cells while the permeate passing though the membrane is substantially free of biocatalyst cells. The biocatalyst enriched fermentation broth from the optional separator, known as the retentate when using membrane filtration as the separation technique, may be recycled to the bioreactor for continued use in the gas fermentation. As used herein, the term “enriched” can mean that the outlet stream has a greater concentration of the indicated component than in the inlet stream to a vessel. As used herein, the term “depleted” can mean that the inlet stream has a greater concentration of the indicated component than in the outlet stream of a vessel.

Along with a solvent, the fermentation broth generated in the bioreactor is passed to a liquid-liquid extraction unit. The biocatalyst depleted fermentation broth from the optional separator may also be passed to the liquid-liquid extraction unit. Liquid-liquid extraction is a technique for transferring a solute dissolved in a first liquid to a second liquid that is essentially immiscible with the first liquid. There is a net transfer of one or more species from one liquid phase into another liquid phase, often from aqueous to organic. The solvent that has become enriched in solute(s) is called extract stream. The feed solution that has become depleted in solute(s) is called the raffinate stream. The solute herein are the products/metabolites of the gas fermentation, and therefore the products/metabolites are extracted into the extract stream. The remaining portions of the fermentation broth or the biocatalyst depleted fermentation broth from the optional separator form the raffinate stream. Therefore, the raffinate stream is enriched in at least water and media and any biocatalyst and the extract stream is enriched in the at least one organic product and solvent and is reduced in water, media, and any biocatalyst.

In an embodiment, the solvent used in the liquid-liquid extraction should have high selectivity between the organic products/metabolites and water to reduce the amount of water carried to downstream processing units. Solvent selectivity refers to the ability of a given solvent to selectively dissolve one compound as opposed to another. Selectivity is measured by comparing the ratio of the weight fraction second liquid to the weight fraction of the first liquid in the extract phase to that in the raffinate phase at equilibrium. The solvent should also have a high distribution coefficient. The distribution coefficient is the concentration of solute in the extract phase to that in the raffinate phase. A high distribution ratio results in the use of a lesser quantity of solvent. Therefore, the solvent may be a higher molecular weight isobutyl ketone, or a blend of a ketone with a highly insoluble hydrocarbon, such as high molecular weight paraffin having at least 10 carbon atoms. In an embodiment, the solvent is selected from methyl isobutyl ketone (MIBK), ethyl isobutyl ketone (EIBK), or any combination thereof. When the product is ethanol, it is desired for the extract stream to at least approach or exceed the water-ethanol azeotrope of about 95 wt. % ethanol. Exceeding the water-ethanol azeotrope may allow elimination of a final azeotropic water-removal step. In another embodiment the solvent may be selected from hexane, ricinoleyl alcohol, methyl ricinoleate, or any combination thereof.

In some liquid-liquid extraction processes, the solvent is removed from the raffinate before recycling to a bioreactor as the solvent may damage the biocatalyst. While such solvent removal may be employed herein, when using solvents MIBK and/or EIBK, the solvent need not be separated from the raffinate before recycling to the bioreactor since these solvents have been found not to be detrimental to envisioned biocatalysts. On the other hand, it is desirable to recover solvent from the extract stream for reuse of the solvent in the liquid-liquid extraction unit. Solvent recovery may be accomplished by techniques such as distillation. The extract stream from the liquid-liquid extraction unit may be passed to a distillation unit where it is separated by distillation into an overhead stream and a bottoms stream. The overhead stream is enriched in at least one organic product and reduced in water and media, and the bottoms stream is enriched in the solvent, water, and media and depleted in the product. In some embodiments, the raffinate stream and at least a first portion of the bottoms stream may be recycled to the bioreactor. At least a second portion of the bottoms stream may be recycled to the liquid-liquid extraction unit to continually provide solvent to the liquid-liquid extraction unit.

In an embodiment, the overhead stream from the distillation unit is passed to a purification unit. The purification unit may employ techniques such as distillation, pressure swing adsorption, or membrane separation. In an embodiment, the purification technique is a process to separate/purify product stream above azeotrope concentration. The overhead stream is separated into a product stream enriched in the at least one organic product and an effluent stream enriched in water, media, and heavy metabolites. In an embodiment, heavy metabolites may be a by-product with a higher boiling point than the desired organic product such as fusel oil. The product stream may be collected to recover at least one organic product. The organic products are the gas fermentation products having from 1 to 18 carbon atoms. For example, organic compounds may include one or more C1 to C4, or C4 to C8, or C8 to C10, or C10 to C12, or C12 to C14, or C14 to C16, or C16 to C18 alcohols and carboxylic acids. In an embodiment, the organic compounds may include one or more C1 to C4 or C6 or C8 or C10 or C12 or C14 or C16 or C18 alcohols and carboxylic acids, C2 to C4 or C6 or C8 or C10 or C12 or C14 or C16 or C18 alcohols and carboxylic acids, C3 to C4 or C6 or C8 or C10 or C12 or C14 or C16 or C18 alcohols and carboxylic acids, C4 to C6 or C8 or C10 or C12 or C14 or C16 or C18 alcohols and carboxylic acids, C6 to C8 or C10 or C12 or C14 or C16 or C18 alcohols and carboxylic acids, C8 to C10 or C12 or C14 or C16 or C18 alcohols and carboxylic acids, C10 to C12 or C14 or C16 or C18 alcohols and carboxylic acids, C12 to C14 or C16 or C18 alcohols and carboxylic acids, C14 to C16 or C18 alcohols and carboxylic acids, C16 to C18 alcohols and carboxylic acids, such as methanol, ethanol, 1-propanol, 2-methyl-1-propanol, 1-butanol, 2-butanol, 3-methyl-1-butanol, isobutanol, or octanol, including but are not limited to acetic acid or lactic acid.

At least a portion of the effluent stream may either be recycled to the bioreactor or may be passed to a wastewater treatment unit or both. A typical wastewater treatment process may include several separate treatment steps such as product removal, anaerobic digestion, and biological oxidation. Such treatment steps serve to remove various components and produce a clarified or treated water stream. The clarified or treated water stream generated from the wastewater treatment unit may be recycled to the bioreactor. Incorporating the upstream liquid-liquid extraction step minimizes the volume of material, specifically wastewater, that is passed to the wastewater treatment process. The less material passed to the wastewater treatment process, the smaller the process and the lower the capital and operation costs. In one embodiment, the volume of wastewater is small enough that it is only treated to the extent to be sent out of the unit as a purge stream. Minimizing the volume of wastewater also results in the savings of acid and base, and other additives which are otherwise recycled to the bioreactor if not removed with water. The amount of wastewater passed to the wastewater treatment unit is minimized by directly recycling water to the bioreactor. For example, up to 67 wt. % of the permeate flow from a membrane filtration separator, including biocatalyst enriched fermentation broth, and bottoms stream from the distillation unit may be recycled directly to the bioreactor, after an initial stripping of product such as ethanol. However, these steps result in the heaviest of the metabolite by-products being concentrated by up to a factor of 3, as compared to the steady-state production rate of these compounds. The optional purification unit discussed above is used to address heavy metabolite by-products.

In an embodiment, the fermentation broth, the extract stream, and the overhead stream further comprise fusel oil. Fusel oil is a mixture of volatile, oily liquids produced in small amounts during alcoholic fermentation. A typical fusel oil contains 60-70 percent of amyl alcohol, smaller amounts of n-propyl and isobutyl alcohols, and traces of other components. The purification unit may additionally provide for the separation of a fusel oil stream.

In another embodiment, the treated water stream, the first portion of the bottoms stream from the distillation unit, or both may comprise an undesired component which may harm the biocatalyst or biocatalyst performance upon recycling to the bioreactor. The undesired component may be a byproduct of the gas fermentation process and present in the gas fermentation broth. Therefore, the first portion of the bottoms stream, the treated water stream, or the combination thereof, may be passed to a removal unit to remove, convert, or reduce the amount of the undesired component before being recycled to the bioreactor. The removal unit may be, for example, an adsorption system such as a guard bed or other solid phase adsorbent. The removal unit may be a catalytic reactor to convert the undesired component to a non-harmful component. In one embodiment, the undesired component may be at least one sulfur compound.

In an embodiment, at least a portion of the biocatalyst enriched fermentation broth may be passed to a drier to recover at least a portion of the biocatalyst as dried spent biocatalyst. This portion of the biocatalyst enriched fermentation broth is sometimes called a bleed stream and can be used as a purge to remove non-viable biocatalyst from the system. The dried spent biocatalyst itself has value as a product due to its high protein content.

By combining the liquid-liquid extraction unit having specific solvents and optimized operational conditions with a distillation unit, at least one organic product can be effectively recovered and separated from fermentation broth all while reducing the amount of water passed to the wastewater treatment and assisting the viability of the microorganisms within the fermentation broth. Additionally, through the optimal configuration of these vessels, the unwanted by-products produced are reduced, energy is conserved, and production of specific target products is maximized.

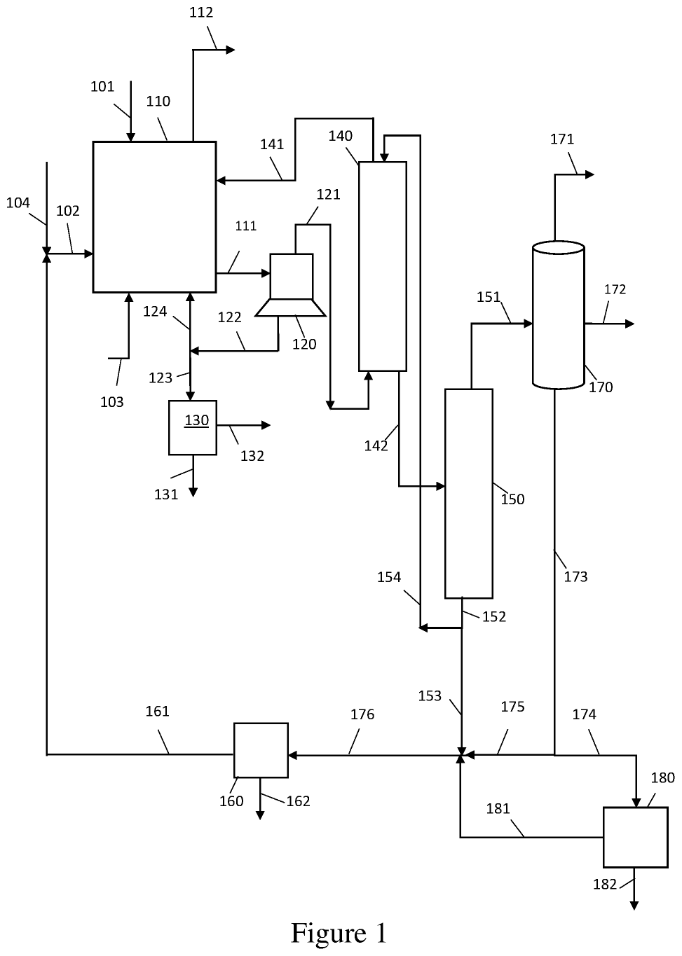

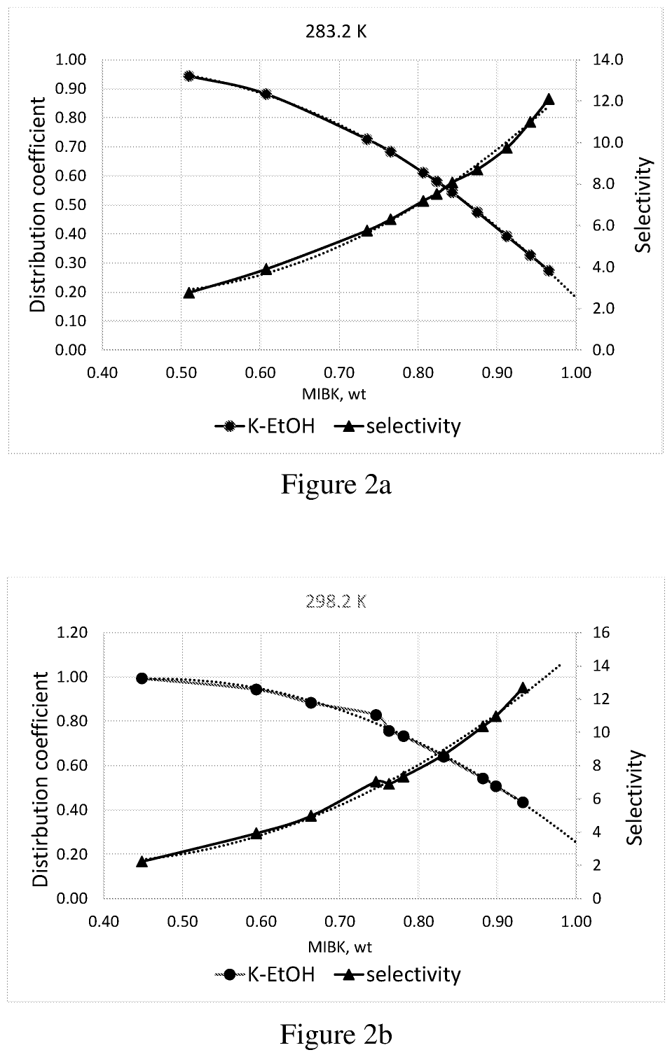

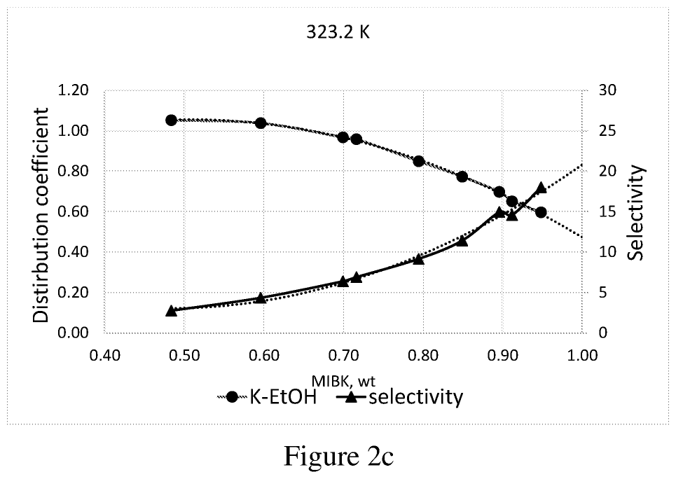

Some embodiments of the disclosure may be described by reference to the process configuration shown in FIGS. 1 and 2, which relates to both apparatus and methods to carry out the disclosure. Any reference to a method step includes reference to an apparatus unit or equipment that is suitable to carry out the step, and vice-versa.

One embodiment of the process and apparatus of the disclosure is described in FIG. 1. However, embodiments of the disclosure and figures are described by way of example, and it should be appreciated that particular steps or stages necessary in one embodiment may not be necessary in another. Conversely, steps or stages included in the description of a particular embodiment can be optionally advantageously used in embodiments where they are not specifically mentioned. For example, the scope includes application to liquid fermentation processes such as sugar-based fermentation processes.

FIG. 1 shows a schematic flow diagram of a continuous system and method, as one embodiment of the disclosure, for recovering at least one organic product from fermentation broth. A substrate steam 103 and/or C1-carbon source, aqueous media stream 101, and water stream 102 are provided to bioreactor 110. Fermentation broth comprising water, media, biocatalyst, and at least one organic product is generated in bioreactor 110. By-product off-gas stream 112 generated in the bioreactor 110 may be removed. The fermentation broth may be passed to separator 120 via fermentation broth conduit 111 to separate the fermentation broth into a biocatalyst depleted fermentation broth stream 121 and a biocatalyst enriched fermentation broth stream 122. The biocatalyst depleted fermentation broth stream 121 is passed to liquid-liquid extraction unit 140 comprising a MIBK solvent. In liquid-liquid extraction unit 140, biocatalyst depleted fermentation broth stream 121 is separated into raffinate stream 141 and extract stream 142 by MIBK solvent. The raffinate stream 141 is enriched in at least water and media and the extract stream 142 is enriched in at least one organic product and MIBK solvent.

Extract stream 142 is passed to distillation unit 150. In distillation unit 150, extract stream 142 is separated into overhead stream 151, and bottoms stream 152. The overhead stream 151 is enriched in at least one organic product and the bottoms stream 152 is enriched in the solvent, water, and media. Raffinate stream 141 and at least a first portion of bottoms stream 153 may be recycled to bioreactor 110. At least a second portion of bottoms stream 154 is passed to the liquid-liquid extraction unit 140 to recycle the MIBK solvent.

Overhead stream 151 may be passed to purification unit 170 where the overhead stream is separated into product stream 171 enriched in at least one organic product, and effluent stream 173 enriched in water and media. Product stream 171 may be collected. Fusel oil stream 172 may also be optionally separated from the overhead stream 151 in the purification unit 170.

At least a portion of effluent stream 174 may be passed to wastewater treatment unit 180 to obtain treated water stream 181. In an embodiment, a small volume of wastewater may be sent out of the wastewater treatment unit 180 as purge stream 182. Gas fermentation may result in the generation of at least one undesirable component in the fermentation broth. Therefore, treated water stream 181, at least a second portion of bottoms stream 153, and effluent stream 175, may be passed separately or in a combined stream 176 to removal unit 160 to remove or reduce the concentration of the undesired component in stream 176 to produce recovered water stream 161 and heavy metabolites stream 162 may be removed. Recovered water stream 161 may be recycled to bioreactor 110 or to water stream 102. A make-up water stream 104 may also be passed to bioreactor 110.

In an embodiment, at least a first stream 123 of biocatalyst enriched fermentation broth 122 is passed to drier 130 while the second stream 124 of biocatalyst enriched fermentation broth 122 is recycled to bioreactor 110. Dried biocatalyst stream 131 is recovered from drier 130 and vapour stream 132 is removed from dryer 130.

Liquid-liquid extraction is the separation of the constituents of a liquid by contact with another insoluble liquid called solvent. The constituents get distributed between the two phases. The solvent rich phase is called extract and the residual liquid from which the solute has been removed is called raffinate. In a liquid-liquid extraction operation generally ternary systems are involved. The equilibrium concentration of such systems is called ternary phase diagrams and may be represented in a triangular coordinate system. As an exemplary embodiment of the present disclosure, FIGS. 2a-2c show the graphs depicting the selectivity and the distribution coefficient (K-EtOH) of ethanol versus composition by weight of the MIBK solvent. These graphs are derived from the ternary phase diagrams of water-ethanol-methyl isobutyl ketone (H₂O-EtOH-MIBK).

The following Tables A, B, and C show data for H₂O-EtOH-MIBK ternary systems at different temperatures:

TABLE A
Data for H₂O-EtOH-MIBK at 10° C.
Temperature: 10° C. (283.2 K)

| MIBK | | | | H₂O | | | | |
|---|---|---|---|---|---|---|---|---|
| MIBK | EtOH | H₂O | Wt % | MIBK | EtOH | H₂O | K-EtOH | Selectivity |
| 0.9827 | 0.0000 | 0.0173 | | 0.0229 | 0.0000 | 0.9771 | | |
| 0.9658 | 0.0135 | 0.0208 | 39.3 | 0.0325 | 0.0492 | 0.9183 | 0.274 | 12.1 |
| 0.9423 | 0.0317 | 0.0260 | 54.9 | 0.0271 | 0.0970 | 0.8759 | 0.274 | 11.0 |
| 0.9127 | 0.0537 | 0.0336 | 61.5 | 0.0264 | 0.1370 | 0.8366 | 0.327 | 9.8 |
| 0.8751 | 0.0812 | 0.0437 | 65.0 | 0.0277 | 0.1708 | 0.8014 | 0.475 | 8.7 |
| 0.8434 | 0.1042 | 0.0524 | 66.5 | 0.0287 | 0.1918 | 0.7795 | 0.543 | 8.1 |
| 0.8238 | 0.1170 | 0.0591 | 66.4 | 0.0317 | 0.2015 | 0.7668 | 0.581 | 7.5 |
| 0.8067 | 0.1289 | 0.0643 | 66.7 | 0.0315 | 0.2109 | 0.7575 | 0.611 | 7.2 |
| 0.7654 | 0.1549 | 0.0797 | 66.0 | 0.0379 | 0.2268 | 0.7353 | 0.683 | 6.3 |
| 0.7354 | 0.1735 | 0.0911 | 65.6 | 0.0385 | 0.2389 | 0.7226 | 0.726 | 5.8 |
| 0.6077 | 0.2421 | 0.1502 | 61.7 | 0.0602 | 0.2745 | 0.6653 | 0.882 | 3.9 |
| 0.5099 | 0.2810 | 0.2091 | 57.3 | 0.0888 | 0.2977 | 0.6134 | 0.944 | 2.8 |

TABLE B
Data for H₂O-EtOH-MIBK at 25° C.
Temperature: 25° C. (298.2 K)

| MIBK | | | | H₂O | | | | |
|---|---|---|---|---|---|---|---|---|
| MIBK | EtOH | H₂O | Wt % | MIBK | EtOH | H₂O | K-EtOH | Selectivity |
| 0.9792 | 0.0000 | 0.0208 | | 0.0170 | 0.0000 | 0.9830 | | |
| 0.9593 | 0.0153 | 0.0254 | 37.6 | 0.0201 | 0.0110 | 0.9689 | 1.394 | 53.1 |
| 0.9327 | 0.0366 | 0.0307 | 54.4 | 0.0212 | 0.0843 | 0.8945 | 0.435 | 12.7 |
| 0.8983 | 0.0623 | 0.0394 | 61.2 | 0.0227 | 0.1230 | 0.8543 | 0.506 | 11.0 |
| 0.8819 | 0.0742 | 0.0438 | 62.9 | 0.0249 | 0.1370 | 0.8381 | 0.542 | 10.4 |
| 0.8320 | 0.1089 | 0.0591 | 64.8 | 0.0296 | 0.1703 | 0.8001 | 0.639 | 8.7 |
| 0.7811 | 0.1418 | 0.0771 | 64.8 | 0.0333 | 0.1936 | 0.7731 | 0.732 | 7.3 |
| 0.7624 | 0.1543 | 0.0833 | 64.9 | 0.0353 | 0.2041 | 0.7606 | 0.756 | 6.9 |
| 0.7460 | 0.1636 | 0.0904 | 64.4 | 0.0346 | 0.1976 | 0.7679 | 0.828 | 7.0 |
| 0.6635 | 0.2099 | 0.1266 | 62.4 | 0.0486 | 0.2378 | 0.7137 | 0.883 | 5.0 |
| 0.5939 | 0.2431 | 0.1630 | 59.9 | 0.0626 | 0.2578 | 0.6797 | 0.943 | 3.9 |
| 0.4485 | 0.2895 | 0.2620 | 52.5 | 0.1204 | 0.2915 | 0.5881 | 0.993 | 2.2 |

TABLE C
Data for H₂O-EtOH-MIBK at 50° C.
Temperature: 50° C. (323.2 K)

| MIBK | | | | H₂O | | | | |
|---|---|---|---|---|---|---|---|---|
| MIBK | EtOH | H₂O | Wt % | MIBK | EtOH | H₂O | K-EtOH | Selectivity |
| 0.9750 | 0.0000 | 0.0250 | | 0.0148 | 0.0000 | 0.9852 | | |
| 0.9731 | 0.0000 | 0.0269 | | 0.0138 | 0.0000 | 0.9862 | | |
| 0.9483 | 0.0202 | 0.0315 | 39.1 | 0.0156 | 0.0338 | 0.9506 | 0.597 | 18.0 |
| 0.9119 | 0.0475 | 0.0407 | 53.9 | 0.0178 | 0.0729 | 0.9093 | 0.652 | 14.6 |
| 0.8958 | 0.0627 | 0.0415 | 60.2 | 0.0217 | 0.0898 | 0.8885 | 0.698 | 14.9 |
| 0.8493 | 0.0928 | 0.0579 | 61.6 | 0.0242 | 0.1201 | 0.8557 | 0.773 | 11.4 |
| 0.7944 | 0.1296 | 0.0760 | 63.0 | 0.0276 | 0.1526 | 0.8199 | 0.849 | 9.2 |
| 0.7163 | 0.1756 | 0.1082 | 61.9 | 0.0377 | 0.1830 | 0.7793 | 0.959 | 6.9 |
| 0.6994 | 0.1839 | 0.1167 | 61.2 | 0.0384 | 0.1900 | 0.7715 | 0.968 | 6.4 |
| 0.5961 | 0.2336 | 0.1703 | 57.8 | 0.0598 | 0.2251 | 0.7151 | 1.038 | 4.4 |
| 0.4837 | 0.2698 | 0.2465 | 52.3 | 0.0966 | 0.2564 | 0.6470 | 1.052 | 2.8 |

Liquid-liquid extraction may be carried out either as a single stage or as a multistage operation. The extract stream and raffinate stream from each stage should be in equilibrium. Multistage operation may be conducted as a cross-current or a counter-current mode of operation. In an embodiment, the multistage system of the liquid-liquid extraction process may be represented as H₂O-EtOH-MIBK ternary phase diagram. The H₂O-EtOH-MIBK ternary phase diagram shows possible phases and their equilibrium according to the composition of a mixture of H₂O-EtOH-MIBK at a constant temperature. FIGS. 2a-2c show graphs of the selectivity and the distribution coefficient (K-EtOH) of ethanol with respect to the composition by weight of MIBK solvent at 10° C. (283.2 K), 25° C. (298.2 K), and 50° C. (323.2 K). The distribution coefficient (K) is the ratio (at equilibrium) of the concentration of solute in the extract and raffinate streams. It gives a measure of the affinity of the solute for the two streams. The distribution coefficient (K-EtOH) of ethanol was approximately 0.3, approximately 0.4, and approximately 0.5 at the temperature of 10° C. (283.2 K), 25° C. (298.2 K), and 50° C. (323.2 K) respectively. The selectivity of ethanol is also increasing with temperature, so it is advantageous to operate the liquid-liquid extraction unit above 50° C. Selectivity advantages may outweigh any additional heating and cooling of the biocatalyst depleted fermentation broth to the liquid-liquid extraction unit.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural unless otherwise indicated herein or clearly contradicted by context. The terms “comprising,” “having,” “including,” and “containing” are to be construed as open-ended terms meaning “including, but not limited to” unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language is intended merely to better illuminate the disclosure and does not pose a limitation on the scope of the disclosure unless otherwise claimed. No language in the specification should be construed as indicating any non-claimed element as essential to the practice of the disclosure.

Variations of embodiments disclosed herein may become apparent to those of ordinary skill in the art upon reading the foregoing description. It is expected skilled artisans to employ such variations as appropriate, and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A process for recovering at least one organic product from fermentation broth comprising:
a. generating, in a bioreactor, a fermentation broth comprising water, media, biocatalyst, and the at least one organic product;
b. separating, using a solvent and a liquid-liquid extraction process, the fermentation broth to generate a raffinate stream and an extract stream, wherein the raffinate stream is enriched in at least water and media and the extract stream enriched is in the solvent and at least one organic product and depleted in water and media;
c. passing the extract stream to a distillation unit and separating into an overhead stream enriched in at least one organic product, and a bottoms stream enriched in the solvent, water, and media;
d. passing the raffinate stream and at least a first portion of the bottoms stream to the bioreactor; and
e. passing at least a second portion of the bottoms stream to the liquid-liquid extraction process.

2. The process of claim 1 further comprising separating, in a purification unit, the overhead stream into a product stream enriched in at least one organic product and an effluent stream enriched in water and media and collecting the product stream.

3. The process of claim 2 further comprising passing at least a portion of the effluent stream to the bioreactor, to a wastewater treatment unit, or to both.

4. The process of claim 3 further comprising generating a treated water stream in the wastewater treatment unit and recycling the treated water stream to the bioreactor.

5. The process of claim 1 wherein the fermentation broth and the extract stream and the overhead stream further comprise fusel oils and separating the overhead stream in the purification unit further comprises separating a fusel oil stream.

6. The process of claim 1 wherein the fermentation broth comprises an undesired component, the process further comprising reducing the amount of the undesired component from the first portion of the bottoms stream before recycling to the bioreactor.

7. The process of claim 1 further comprising separating at least a portion of the biocatalyst from the fermentation broth before separating the fermentation broth by liquid-liquid extraction.

8. The process of claim 7 further comprising recycling at least a portion of the separated biocatalyst to the bioreactor, drying and recovering at least a portion of the separated biocatalyst, or both.

9. The process of claim 1 wherein the solvent is methyl isobutyl ketone, ethyl isobutyl ketone, or any combination thereof.

10. The process of claim 1 wherein the solvent is hexane, ricinoleyl alcohol, methyl ricinoleate, or any combination thereof.

11. The process of claim 1 wherein the at least one organic product is selected from C1 to C18 alcohols and carboxylic acids.

12. An apparatus for recovering at least one organic product from fermentation broth comprising:
a. a bioreactor comprising at least a fermentation broth conduit;
b. a liquid-liquid extraction unit in fluid communication with the fermentation broth conduit, the liquid-liquid extraction unit comprising a raffinate conduit and an extract conduit; and
c. a distillation unit in fluid communication with the extract conduit, the distillation unit comprising a bottoms conduit in fluid communication with the liquid-liquid extraction unit, and an overhead conduit.

13. The apparatus of claim 12 further comprising a purification unit in fluid communication with the overhead conduit, the purification unit comprising a product conduit, an effluent conduit, and a fusel oil conduit.

14. The apparatus of claim 12 further comprising a separator in fluid communication with the fermentation broth conduit and the liquid-liquid extraction unit.

15. The apparatus of claim 12 further comprising a wastewater treatment unit in fluid communication with the effluent conduit.

16. The apparatus of claim 12 further comprising a removal unit in fluid communication with at least one of the wastewater treatment unit, the effluent conduit, or the bottoms conduit.

17. The apparatus of claim 16 wherein the removal unit is further in fluid communication with the bioreactor.

18. The apparatus of claim 12 further comprising a drier in fluid communication with the bioreactor.

19. The apparatus of claim 12 wherein the raffinate conduit is in fluid communication with the bioreactor.

20. The apparatus of claim 12 wherein the bioreactor further comprises at least one substrate conduit, at least one media conduit, and an off-gas conduit.
